(19) United States
(12) Patent Application Publication
(10) Pub. No.: US 2003/0035791 A1
(43) Pub. Date: Feb. 20, 2003

(54) Modulation of p57kip2 expression and uses thereof in the treatment of diabetes and hyperinsulinism of infancy

(76) Inventors: Benjamin Glaser, Jerusalem (IL); Samir Kassem, Moqebla (IL); Ilana Ariel, Jerusalem (IL)

Correspondence Address: Cooper & Dunham LLP, 1185 Avenue of the Americas, New York, NY, 10036, US

(21) Appl. No.: 09/919,398

(22) Filed: Jul. 31, 2001

(51) Int. Cl. A61K 48/00; C12N 15/86

(52) U.S. Cl. 424/93.2; 435/456; 514/44

(57) ABSTRACT

The present invention relates to in vivo and in vitro methods for controlling proliferation of glucose regulated insulin-producing beta cells by modulating the expression or the activity of the cyclin-dependent kinase inhibitor p57Kip2. The invention further provides recombinant glucose regulated insulin-producing beta cell or cell-line having controlled proliferation and compositions and uses thereof in methods for treatment of diabetes type I, diabetes type II and hyperinsulinism of infancy.

### FIELD OF THE INVENTION

The present invention relates to methods for treatment of diabetes type I, diabetes type II and hyperinsulinism of infancy. More particularly, the present invention relates to a method for controlling proliferation of glucose regulated insulin-producing beta cells by modulating the expression or the activity of the cyclin-dependent kinase inhibitor p57Kip2.

### BACKGROUND OF THE INVENTION

Hyperinsulinism of infancy (HI) is a rare genetic disorder with a prevalence in out bred populations of about 1/50,000 live births [Bruining G J, et al., Cur Op Pediatr 2:758-765 (1990); Otonkoski T., et al., Diabetes 48:408-15 (1999)]. An incidence as high as 1:2,500 has been reported in inbred populations [Otonkoski T., et al., ibid. (1999); Mathew P M, Clin Pediatr Phila 27:148-51 (1988)]. The molecular basis of the disease was recently elucidated, and most cases are caused by mutations in either the sulfonylurea receptor gene (SUR1, ABCC8) or the inward rectifying potassium channel gene (Kir6.2, KCNJ11), the two subunits of the beta-cell KATP channel [Nestorowicz A, et al., Hum Molec Genet 5:1813-1822 (1996); Nestorowicz A, et al., Diabetes 46:1743-1748 (1997); Thomas P M, et al., Am J Hum Genet 56:416-421 (1995); Thomas P, et al., Hum Mol Genet 5:1809-1812 (1996)]. A minority of patients have glucokinase (GK) or glutamate dehydrogenase (GLUD-1) mutations, whereas in 40-50% of the patients the genetic cause of the disease is still not known [Nestorowicz A, ibid. (1997); Glaser B, et al., New Engl J Med 338:226-230 (1998); Stanley C A, Diabetes 49:667-73 (2000); Nestorowicz A, et al., Hum Mol Genet 7:1119-1128 (1998)].

The clinical presentation of HI can be variable, ranging from mild disease to severe, life-threatening hypoglycemia, which, if not adequately treated, causes irreversible neurological damage [Aynsley-Green A, et al., Dev Med Child Neurol 23:372-9 (1981); Landau H, et al., Pediatrics 70:440-6 (1982)].

The histological appearance of pancreases from affected children is heterogenous, and can be subdivided into 2 major forms, diffuse-HI and focal-HI [Rahier J, et al., Diabetologia 26:282-9 (1984); Lab Invest 42:356-65 (1980); Goossens A, et al., Am J Surg Pathol 13:766-75 (1989)]. The diffuse form is more common and bears some histological characteristics of nesidioblastosis, a normal phenomenon observed in the fetus and newborn which includes poorly defined islets, small clusters of endocrine cells scattered throughout the exocrine tissue, and a high frequency of endocrine cells interposed between ductular cells [Goossens A, et al., ibid. (1989); Jaffe R, et al., Perspect Pediatr Pathol 7:137-65 (1982); Rahier J, et al., Diabetologia 20:540-6 (1981)].

Focal-HI can generally be recognized as a discrete region of adenomatous hyperplasia often too small to be identified macroscopically. Histologically the lesion is comprised of nodules of endocrine and exocrine elements. The beta-cells are pleomorphic, some having giant nuclei and abundant cytoplasm [Rahier J, ibid. (1984)]. The rest of the pancreas has normal endocrine architecture for age with beta-cells containing small nuclei and shrunken cytoplasm [Rahier J, et al., Histopathology 32:15-9 (1998)].

The inventors have previously reported increased frequencies of proliferating beta-cells in pancreases from HI patients and in early stages of human development. Focal-HI presented the highest proliferation frequency compared to diffuse-HI and controls [Kassem S A, et al., Diabetes 49:1325-33 (2000)]. The mechanisms regulating the rate of beta-cell proliferation are not known, however, the genetic alteration in focal-HI may provide an insight into the control of beta-cell turnover.

Focal-HI is caused by the somatic loss of part of the short arm of maternal chromosome 11 in a beta-cell precursor of a patient carrying a mutant SUR-1 gene on the paternal allele [Fournet J C, et al., Annales D Endocrinologie 59:485-91 (1998); Ryan F D, et al., Arch Dis Child 79:445-447 (1998)]. In all cases it is the paternal allele that carries the mutation and the maternal allele that is somatically lost, suggesting that the gene (or genes) responsible for the focal proliferation is imprinted. A large number of genes are located in the lost portion Ch11p, including p57^{KIP2}, H19, Insulin-like Growth Factor II (IGF-II) and p53-induced protein with a death domain (Pidd). Pidd is a 910 amino acid protein induced by the tumor suppressor p53 that promotes apoptosis [Lin Y, et al., Nat Genet 26:122-7 (2000)]. It is not known if this gene is imprinted or if it is expressed in beta-cells. IGF-II is imprinted with only the paternal allele expressed, and increased expression of this gene has been associated with increased beta-cell proliferation and overgrowth syndromes [Petrik J, et al., Endocrinology 140:2353-63 (1999); Petrik J, et al., Endocrinology 139:2994-3004 (1998)]. Both p57^{KIP2} and H19 are paternally imprinted, with only the maternal allele expressed and thus are candidate genes for enhanced cell proliferation [Rachmilewitz J, et al., Febs Letters 309: 25-8 (1992); Matsuoka S, et al., Genes And Development 9:650-62 (1995); Matsuoka S, et al., Proc Natl Acad Sci USA. 93:3026-30 (1996); Lee M H, Genes Dev 9:639-49 (1995)]. H19 is an untranslated RNA molecule thought to be an important regulator of IGF-II mRNA levels [Li M, et al., Clinical Genetics 53:165-70 (1998)]. p57^{KIP2} (CDKN1C) is a 1.5 kb gene encoding a 335 amino acid peptide that belongs to the cyclin-dependent kinase (Cdk) inhibitor family. It is an important inhibitor of several G1 cyclin/Cdk complexes causing cell cycle arrest in terminally differentiated cells [Matsuoka S, et al., Genes. Dev. 9:650-62 (1995); Lee M H, et al., Genes Dev 9:639-49 (1995)], and loss or underexpression of p57^{KIP2} has been related to several malignancies [Kondo M, et al., Oncogene 12:1365-8 (1996); Bourcigaux N, et al., J Clin Endocrinol Metab 85:322-30 (2000); Thompson J S, et al., Cancer Res 56:5723-7 (1996)]. It is not known if p57^{KIP2} is expressed or imprinted in human beta-cells.

Using immunohistochemistry the inventors examined normal pancreases from different developmental stages and pancreases from patients with both diffuse- and focal-HI for p57^{KIP2} expression. Using immunofluorescence and computerized imaging, a method to quantify IGF-II staining in beta-cells was developed. The inventors observations suggest a cell-specific localization of p57^{KIP2} and IGF-II in beta-cells. A stable fraction of beta-cells expressed p57^{KIP2} during different developmental stages. The inventors have demonstrated loss of p57KIP2 inside lesions of focal HI, a finding consistent with increased rates of proliferation previously demonstrated. IGF-II expression inside the focal lesions was mildly increased when compared to the beta-cells in the unaffected surrounding tissue.

It is thus an object of the invention to provide a method for controlling proliferation of glucose regulated insulin-producing beta cells by modulating the expression of the cyclin-dependent kinase inhibitor p57Kip2.

### SUMMARY OF THE INVENTION

The present invention relates to a method for controlling proliferation of glucose regulated insulin-producing beta cells by modulating the expression or the activity of the cyclin-dependent kinase inhibitor p57Kip2. Controlling the proliferation of the beta cells according to the invention comprises the step of transforming the cells with an expression vector comprising the sense, the antisense or, alternatively, mutated nucleic acid sequence of the p57Kip2.

In a preferred embodiment, the expression vector used by the method of the invention further comprises an inducible promoter, a beta-cell specific transcriptional regulating sequence and optionally operably linked additional control, promoting and/or other regulatory elements. The expression vector according to the invention may be a plasmid or virus.

In a specifically preferred embodiment, modulation of the expression or activity of p57Kip2 according to the invention may be down-regulation or up-regulation of p57Kip2 expression or activity. More specifically, down-regulation of p57Kip2 expression may be achieved by transforming said cells with an expression vector comprising an anti-sense nucleic acid sequence directed against the nucleic acid sequence encoding p57Kip2 and optionally an inducible promoter. Down-regulation of p57Kip2 activity may be achieved by transforming said cells with an expression vector comprising a mutated nucleic acid sequence of p57Kip2 and optionally an inducible promoter. Such down-regulation of p57Kip2 expression or activity results in increased proliferation of the transformed cell.

Alternatively, in case decreased proliferation of beta-cells is desired, over-expression of p57Kip2 is to be adopted. Up-regulation of p57Kip2 may be achieved by transforming said cells with an expression vector comprising the sense nucleic acid sequence of the p57Kip2 and optionally an inducible promoter.

In yet another particularly preferred embodiment, the present invention relates to a method for the in vivo or ex vivo expansion of glucose regulated insulin-producing beta cells by down-regulation of p57Kip2 expression or activity. This method comprises the step of transforming the cells with an expression vector comprising an antisense nucleic acid sequence directed against the nucleic acid sequence encoding p57Kip2, or with an expression vector comprising a mutated nucleic acid sequence of p57Kip2 and therefore down-regulation of the p57Kip2 expression or activity and increased cell proliferation.

A second aspect of the present invention relates to a recombinant glucose regulated insulin-producing beta cell or cell-line, transformed with an expression vector comprising the sense, antisense or mutated nucleic acid sequence of p57Kip2. The recombinant beta-cell or cell-line according to the present invention has controlled proliferation rate.

In a preferred embodiment, the transformed beta-cell or cell-line according to this aspect of the invention may be a mammalian beta-cell.

In a specifically preferred embodiment, the beta-cell according to the invention is transformed with an expression vector of the invention that further comprises an inducible promoter, a beta-cell specific transcriptional regulating sequence and optionally operably linked additional control, promoting and/or other regulatory elements. This expression vector may be a plasmid or a virus.

The beta-cell of the invention, can express of p57Kip2 in a modulated fashion. This modulation, may be down-regulation or up-regulation of the p57Kip2 expression or activity.

In a specifically preferred embodiment, the expression of p57Kip2 is down regulated in the beta-cells of the invention. Accordingly, down-regulation of p57Kip2 expression may be achieved by transforming these cells with an expression vector comprising an anti-sense nucleic acid sequence directed against the nucleic acid sequence encoding p57Kip2. Down-regulation of p57Kip2 activity may be achieved by transforming these cells with an expression vector comprising a mutated nucleic acid sequence of p57Kip2. These expression vectors may optionally further include an inducible promoter. Thus, under suitable conditions, such transformed cell would proliferate at an increased rate.

According to an alternative preferred embodiment, the expression of p57Kip2 is up-regulated in the beta-cells of the invention. Up-regulation of p57Kip2 expression may be achieved by transforming the cells with an expression vector comprising the sense nucleic acid sequence of the p57Kip2 and optionally an inducible promoter. Thus, such transformed cells would have decreased proliferation.

In a further aspect, the present invention relates to a pharmaceutical composition for modulation of p57Kip2 expression or activity. Such composition comprises as an active ingredient a therapeutically effective amount of any one of the transformed beta-cells of the invention or of expression vector comprising the sense, the antisense or mutated nucleic acid sequence of p57Kip2. The composition of the invention may further comprise pharmaceutically acceptable carriers.

In case down-regulation of p57Kip2 expression is desired, the composition of the invention may comprise as an effective ingredient an anti-sense nucleic acid sequence directed against the nucleic acid sequence encoding p57Kip2 and optionally an inducible promoter, or cells transformed with an expression vector comprising the same. For down-regulation of p57Kip2 activity, the composition of the invention may comprise as an effective ingredient a mutated nucleic acid sequence of p57Kip2 or cells transformed with an expression vector comprising the same.

Alternatively, when up-regulation of p57Kip2 expression is desired, the composition of the invention may comprise as an effective ingredient the nucleic acid sequence encoding p57Kip2 (sense) and optionally an inducible promoter, or cells transformed with an expression vector comprising the same.

According to a preferred embodiment, the pharmaceutical composition of the invention is intended for the treatment of any one of diabetes type I and diabetes type II.

The present invention further provides the use of a recombinant glucose regulated insulin-producing beta cell, transformed with an expression vector comprising an antisense nucleic acid sequence directed against the nucleic acid sequence encoding p57Kip2 or a mutated nucleic acid sequence of p57Kip2, in the preparation of pharmaceutical composition for the treatment of diabetes type I and/or diabetes type II.

In another specific embodiment, the invention relates to the use of an expression vector comprising the sense, the antisense or a mutated nucleic acid sequence encoding p57Kip2, in the preparation of a pharmaceutical composition for the treatment of diabetes type I, diabetes type II and/or disorders associated with increased beta-cell proliferation.

In yet a further aspect, the present invention relates to a method for the treatment of diabetes type I and/or diabetes type II, in a subject having dysfunctional pancreatic beta-islet cells. According to this aspect, the method of the invention comprises administering to the subject in need, a therapeutically effective amount of a recombinant glucose regulated insulin-producing beta cells or of a combination comprising the same. These recombinant cells are transformed with an expression vector comprising an antisense nucleic acid sequence directed against the nucleic acid sequence encoding p57Kip2. Alternatively, these recombinant cells may be transformed with an expression vector comprising a mutated nucleic acid sequence of p57Kip2.

In another specifically preferred embodiment, the method of treatment of diabetes type I and/or diabetes type II, in a subject having dysfunctional pancreatic beta-islet, comprises administering to said subject a therapeutically effective amount of an expression vector comprising an antisense nucleic acid sequence directed against the nucleic acid sequence encoding p57Kip2 or a mutated p57Kip2 sequence, for down-regulation of p57Kip2 expression or activity, and optionally an inducible promoter or of composition comprising the same.

According to a further particular embodiment, the invention relates to a method for treatment of a disorder characterized in increased beta-cell proliferation in a subject. This method comprises administering to a subject in need, a therapeutically effective amount of an expression vector comprising the nucleic acid sequence encoding p57Kip2 or of a pharmaceutical composition comprising the same. This expression vector directs up-regulation of p57Kip2 expression, resulting in decreased proliferation of the target beta-cells.

In a preferred embodiment, the method of the invention is intended for treating a mammalian, preferably, a human subject.

The present invention further provides a method for ex-vivo treating an individual suffering from diabetes type I and/or diabetes type II. Such method comprises the steps of: (a) providing an expression vector comprising an antisense nucleic acid sequence directed against the nucleic acid sequence encoding p57Kip2 for modulation of p57Kip2 expression or an expression vector comprising a mutated nucleic acid sequence of p57Kip2 for modulation of p57Kip2 activity; (b) obtaining cells from an in individual suffering from diabetes type I or diabetes type II, and optionally culturing said cells under suitable conditions; (c) transforming the cells obtained in step (b) with the expression vector provided in (a); (d) in vitro expanding said transformed cells under suitable conditions; and (e). re-introducing said cells obtained in (d) into said individual.

Alternatively, expansion of the transformed beta-cells may be performed in-vivo, under certain conditions suitable for inducing the expression of the anti sense nucleic acid sequence directed against the nucleic acid sequence encoding p57Kip2 or of the mutated nucleic acid sequence of p57Kip2.

### BRIEF DESCRIPTION OF THE FIGURES

FIGS. 1A-L: p57^{KIP2} and IGF-II expression in pancreas

1A: Low power (×100) image of adult pancreas stained for p57^{KIP2} (brown nuclear stain) and insulin (red cytoplasmic stain). 1B-1D: Adult islets (×400) stained for p57^{KIP2} (brown nuclear stain) and insulin, glucagon and somatostatin respectively (red cytoplasmic stain). 1E-1H: Islets stained (×400) for p57^{kip2} and insulin from 26 week fetus (E), 6 week old patient with diffuse-HI (F), and a 5 week old patient with focal HI showing stain outside the lesion (G) and inside the focal lesion (H). p57^{KIP2} positive nuclei are indicated with arrows. I-L: Immunofluorescent staining (×400) for insulin (I, K) and IGF-II (J, L) in focal-HI, outside (I, J) and inside (K, L) the lesion.

FIG. 2: Percent of different islet cell types positive for p57^{KIP2}

The number of samples in each group is given above each column. PP cells were very rare and only in 2 samples was it possible to count 1000 PP positive cells.

FIG. 3: Expression of p57^{KIP2} in different age groups

Percent of beta-cells staining positive for p57^{KIP2} in different age groups, each column represents the mean of 3 samples.

FIG. 4: Percent of beta-cells staining positive for p57^{KIP2} in controls, diffuse-HI and focal-HI

In focal disease, beta-cells outside the lesion and within the lesion were evaluated separately. The number of samples in each group is given above each column.

FIG. 5: IGF-II expression in focal-HI inside and outside the lesion

IGF-II expression in focal-HI inside and outside the lesion (n=8), expressed as a ratio of the integrated optical density of IGF-II staining divided by the insulin stained area.

### DETAILED DESCRIPTION OF THE INVENTION

A number of methods of the art of molecular biology are not detailed herein, being well known to the person of skill in the art. Such methods include site-directed mutagenesis, PCR cloning, expression of cDNAs, analysis of recombinant proteins or peptides, transformation of bacterial and yeast cells, transfection of mammalian cells, and the like. Textbooks describing such methods are e.g., Sambrook et al., Molecular Cloning A Laboratory Manual, Cold Spring 10 Harbor Laboratory; ISBN: 0879693096, 1989, Current Protocols in Molecular Biology, by F. M. Ausubel, ISBN: 047150338X, John Wiley & Sons, Inc. 1988, and Short Protocols in Molecular Biology, by F. M. Ausubel et al. (eds.) 3rd ed. John Wiley & Sons; ISBN: 0471137812, 1995. These publications are incorporated herein in their entirety by reference. Furthermore, a number of immunological techniques are not in each instance described herein in detail, as they are well known to the person of skill in the art. See e.g., Current Protocols in Immunology, Coligan et al. (eds), John Wiley & Sons. Inc., New York, N.Y.

The p57^{KIP2} protein, originally described in 1995, is a cyclin-dependent kinase (Cdk) inhibitor causing cell cycle arrest and accumulation of cells in the G1-phase. It has been shown to bind to cyclin/Cdk complexes in a cyclin-dependent manner and inhibit their activity [Matsuoka S, et al. Genes Dev 9:650-62, (1995); Lee M H, et al., Genes Dev 9:639-49 (1995)]. The gene is located within a cluster of imprinted genes in humans and mice, with the parental allele mainly expressed [Matsuoka S, et al., Proc Nat Acad Sci USA 93:3026-30 (1996); Hatada I, et al., Nat Genet 11:204-6, (1995)].

The present inventors demonstrated that, in the pancreas, p57KIP2 is expressed almost exclusively in the endocrine cells and within the islets, expression being primarily localized to beta-cells. During development, the proportion of beta-cells expressing p57^{KIP2} does not appear to vary, and also in diffuse Hyperinsulinism of Infancy (HI), the proportion of beta-cells expressing the protein is not different from controls of a similar age group. In contrast, p57^{KIP2} is not expressed by beta-cells within the focal-HI lesion. IGF-II expression was also seen primarily in beta-cells and staining was increased within the lesion of focal-HI when compared to beta-cells in the unaffected region.

The finding that p57^{KIP2} expression is limited to the endocrine portion of the pancreas explains the low gene expression reported in human whole-pancreas mRNA preparations [Matsuoka S, et al. ibid. (1995); Lee M H, et al., ibid. (1995)]. It is also consistent with the nature of the islet cell population, especially beta-cells, being post-mitotic and terminally differentiated. This may partially account for failure of beta cell regeneration following exposure to harmful factors such as hyperglycemia and hyperlipidemia, a phenomenon that may have important implications in the pathogenesis of type II diabetes mellitus. The low expression in other islet cells compared to beta cells suggests that the latter represent a higher differentiation stage. The very low p57^{KIP2} expression in acinar and ductular cells may provide a possible explanation for the proliferative capacity those cells retain [Bouwens, L, Microsc Res Tech 43:332-6 (1998)].

The surprising association of decreased expression of the p57Kip2, with proliferating cells located within the lesions area of focal HI and the specificity of this phenomenon to beta-cells, led the inventors to develop methods for controlling beta-cell proliferation by modulation of p75Kip2 expression.

Thus, as a first aspect, the present invention relates to a method for controlling proliferation of glucose regulated insulin-producing beta cells by modulating the expression or the activity of the cyclin-dependent kinase inhibitor p57Kip2. Controlling the proliferation of the beta cells according to the invention, comprises the step of transforming the cells with an expression vector comprising the sense or, alternatively, the antisense nucleic acid sequence of the p57Kip2. Modulation of p57Kip2 activity may be performed according to the present invention by transforming the cells with an expression vector comprising a mutated nucleic acid sequence of p57Kip2. Preferred examples for such mutants according to the invention, would be a dominant negative mutant of p57Kip2.

It is to be appreciated that different biological or chemical agents which may further modulate the expression or the activity of p57Kip2, are within the scope of the present invention.

Expression vectors for production of the molecules of the invention include plasmids, phagemids or other vectors. “Vectors”, as used herein, encompass plasmids, viruses, bacteriophage, integratable DNA fragments, and other vehicles, which enable the integration of DNA fragments into the genome of the host. Expression vectors are typically self-replicating DNA or RNA constructs containing the desired nucleic acid sequence or its fragments, and operably linked genetic control elements that are recognized in a suitable host cell and effect expression of the desired genes within the host. Generally, the genetic control elements can include a prokaryotic promoter system or a eukaryotic promoter expression control system. Such system typically includes a transcriptional promoter, an optional operator to control the onset of transcription, transcription enhancers to elevate the level of RNA expression, a sequence that encodes a suitable ribosome binding site, RNA splice junctions, sequences that terminate transcription and translation and so forth. Expression vectors usually contain an origin of replication that allows the vector to replicate independently of the host cell.

A vector may additionally include appropriate restriction sites, antibiotic resistance or other markers for selection of vector containing cells. Plasmids are the most commonly used form of vector but other forms of vectors which serves an equivalent function and which are, or become, known in the art are suitable for use herein. See, e.g., Pouwels et al. [Cloning Vectors: a Laboratory Manual (1985 and supplements), Elsevier, N.Y] and Rodriquez, et al. (eds.) [Vectors: a Survey of Molecular Cloning Vectors and their Uses, Buttersworth, Boston, Mass (1988)], which are incorporated herein by reference.

In general, such vectors contain in addition specific genes, which are capable of providing phenotypic selection in transformed cells. The use of prokaryotic and eukaryotic viral expression vectors to express the nucleic acid sequence of the present invention are also contemplated. These vectors may further contain tagging sequences which are capable of providing convenient isolation of the desired expressed sequences. Such tagging sequences are well known in the art and include for example FLAG, HA, His-6 (six histidine) and the myc tag.

The vector is introduced into a host cell by methods known to those of skilled in the art. Introduction of the vector into the host cell can be accomplished by any method that introduces the construct into the cell, including, for example, transfections by calcium phosphate precipitation, microinjection, electroporation or transformation. See, e.g., Current Protocols in Molecular Biology, Ausuble, F. M., ed., John Wiley & Sons, N.Y. (1989). As used herein, the term “transfection” means the introduction of a nucleic acid, e.g., the DNA construct or an expression vector, into a recipient cells by nucleic acid-mediated gene transfer. “Transformation”, as used herein, refers to a process in which a cell's genotype is changed as a result of the cellular uptake of exogenous DNA or RNA.

As used herein, the term “nucleic acid” refers to polynucleotides such as deoxyribonucleic acid (DNA), and, where appropriate, ribonucleic acid (RNA). The terms should also be understood to include, as equivalents, analogs of either RNA or DNA made from nucleotide analogs, and, as applicable to the embodiment being described, single-stranded (such as sense or antisense) and double-stranded polynucleotides. Antisense nucleic acid sequence is the complementary nucleic acid sequence of a certain coding sequence. Preferably, the antisense nucleic acid sequence used in the present invention is the sequence complementary to the full length or to any fragment of the p57Kip2 coding sequence (sense). The antisense nucleic acid sequence prevents expression (translation) of the sense p57Kip2 strand by complementarily hybridizing to said sense strand and preventing accession to the translation machinery.

The present invention also provides expression vectors containing the sense, the antisense or mutated nucleic acid sequences of the p57Kip2, operably linked to at least one transcriptional regulatory sequence.

In a preferred embodiment, the expression vector used by the method of the invention further comprises an inducible promoter, a beta-cell specific transcriptional regulating sequence and optionally operably linked additional control, promoting and/or other regulatory elements. This expression vector according to the invention may be a plasmid or virus.

The term “operably linked” is used herein for indicating that a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein-coding regions, in the same reading frame.

The inducible promoters and genetic elements in the vectors inducibly regulate the p57Kip expression or creation of the antisense, since the expression or prevention of p57Kip expression, in primary cells would be likely to control their proliferation and therefore may interfere with the ability to differentiate.

Inducible promoters and inducible genetic elements are known in the art and can be derived from viral or mammalian genomes. Numerous examples of inducible promoters are known in the art, including lacO-containing SV40 promoter, lacO-containing LTR promoter, methallothionein promoter and the TET promoter. There are numerous sources of SV40 DNA, including commercial vendors such as New England Biolabs, Inc., Beverly, Mass., USA.

In this system, the inducible promoter may be used to gradually reduce the transcription of the sense or the antisense sequences of p57Kip, e.g., to gradually adapt the cells to the absence or presence of the p57Kip protein.

It is to be appreciated that inducible genetic elements may be used as well. When these elements are used, the p57Kip sense, the antisense or the mutated sequences are excised from the transfected cells. Typically, these genetic elements are recombination sites and introduction or activation of site specific recombinase results in precise excision of the genetic material between the genetic elements.

Construction of suitable vectors containing the desired p57Kip sense, antisense or mutated sequence and inducible promoter and/or genetic elements system employs standard ligation techniques. Isolated plasmids or nucleotide sequences are cleaved, tailored, and religated in the form desired to form the plasmid required. For example, useful plasmid vectors for amplifying the retroviral genetic elements in bacterial hosts prior to transfection are constructed by inserting the desired nucleic acid sequence, the inducible promoter or genetic elements in a vector including one or more phenotypic selectable markers and origin of replication to ensure ampliphicatio within a bacterial host.

Viral vectors such as recombinant viruses can be used to transfect or infect cells, and genetically modified cells selected by using methods known in the art, see e.g., Sambrook, J, et al., eds., Molecular Cloning: A Laboratory Manual, Cold Spring Harbor Laboratory Press, 2d ed. (1989). The genetically modified cells are cultured in conventional nutrient media modified as appropriate for activating or repressing the promoters, and selecting for genetically modified cells. The culture conditions are those suitable for the target cells and will be apparent to those skilled in the art.

A number of different systems have been used to effect inducible function. If the genetically modified cells are to be transplanted, the inducible promoters must not be induced by condition exist in the transplant host, such as the chemicals present in the host or the in vivo environment of the host.

Temperature sensitive mutants of SV40 have been used to effect inducibly transformed cell lines which have been transplanted in vivo and shown to differentiate and retain some normal functions [Chou, J Y, Mol Endocrinol, 3:1511-14 (1989)].

Another possible inducible system is to construct a fusion product between the sense or antisense sequences and a steroid hormone receptor. This has been shown to result in steroid inducible function of the fusion partner. If the genetically modified cells are to be transplanted, the endogenous steroid level of the transplant host must not induce transcription of the sense or the antisense p57Kip.

Examples for other inducible promoters are metallothionein promoter, inducible by heavy metals [Mayo, K E, et al., Cell, 29:99-108 (1982)], the mouse mammary tumor virus (MMTV) promoter, induced by glucocorticoid [Beato, M, et al., J. Steroid Biochem, 27:9-14 (1987)], the TET promoter which is repressed by tetracycline [Pescini, R, et al., Biochem & Biophy Res Communications 202(3):1664-7 (1994)] and the lac repressor-lac operator inducible promoter system. This E. coli system, based on the DNA binding protein namely lac repressor (lacI), and the lac operator (lacO), has been shown to function in mammalian cells [Brown, M, et al., Cell, 49:603-12 (1987)].

As used herein, the term “specific transcriptional regulatory sequence” means a DNA sequence that serves as an promoter or an enhancer, which regulates expression of a selected DNA sequence operably linked thereto, and which affects expression of the selected DNA sequence in specific cells.

The endocrine pancreas of mammals is composed of several thousand islets of Langherhans. Each individual islet contains four hormone-producing cell types in a characteristic proportion and distribution, with the different horomone-producing cells appearing sequentially during embryo genesis [Pictet et al. in Steiner, D F and Frenkel, M (EDS.), Handbook of Physiology, Series 7, American Physiology Society, Washington, D.C., pp. 25-66 (1972); Yoshinari et al., Anat Embryol 165:63-70 (1992); Titelman et al., Dev Biol 121:454-466 (1987); Herrera et al., Development 113:1257-1265 (1991); Gitts et al., PNAS 89:1128-1132 (1992)]. Although the precise lineage relationship between the different islet cells is not known, co-expression of different hormone genes during normal pancreas development and in cloned cell-lines derived from islet cell tumors suggests a common precursor for the pancreatic endocrine cells [Medsen et al., J Cell Biol 103:2025-2034 (1986); Alpert et al., Cell 53:295-308 (1988); Herrera et al. ibid. (1991)]. These observations have suggested that terminal differentiation, restricting the expression of the hormone genes to the individual endoctrine cell-type, occur relatively late in ontogeny of the endocrine pancreas.

For some of these hormone genes it has been possible to identify the cis- and trans-acting elements that regulate the islet-specific expression of the genes. For instance, the insulin-1 gene contains approximately 350 basepairs of 5′ flanking DNA (e.g., the “insulin transcriptional regulatory sequence”) which is sufficient for selective, β-cell specific expression both in cell lines and in transgenic animals [Walker et al. Nature 306:557-581 (1983); Alpert et al., ibid. (1988)], with both a strong β-cell enhancer and a promoter element contained within these 350 base pairs (bp) [Edlund et al., Science 230:912-916 (1983); Karlson et al., PNAS 84:8819-8823 (1987)].

Different transcriptional regulatory sequence such as the 350 bp of insulin gene or any other beta-cell specific enhancer, may serve in the expression vectors of the present invention to direct specific expression of the anti-sense, the sense or the mutated nucleic acid sequences of p57Kip2.

Accordingly, the term control and regulatory elements includes promoters, terminators and other expression control elements. Such regulatory elements are described in Goeddel [Goeddel, Gene Expression Technology: Methods in Enzymology 185, Academic Press, San Diego, Calif. (1990). For instance, any of a wide variety of expression control sequences, which are sequences that control the expression of a DNA sequence when operatively linked to it, may be used in these vectors to express DNA sequences encoding the sense or the antisense sequence of the p57Kip2 protein or any other desired protein located on Ch11p described in the Examples of the present application.

In a specifically preferred embodiment, modulation of the expression of p57Kip2 according to the invention may be down-regulation or up-regulation of the p57Kip2 expression or activity. More specifically, down-regulation of p57Kip2 expression may be achieved by transforming said cells with an expression vector comprising an antisense nucleic acid sequence directed against the nucleic acid sequence encoding p57Kip2 and optionally an inducible promoter. Such down-regulation of p57Kip2 expression results in increased proliferation of the transformed cell. Enhanced proliferation of beta-cells may be also achieved by down-regulation of p57Kip2 activity. Therefore, the present invention further provides a method for down-regulating the p57Kip2 activity by transforming said cells with an expression vector comprising a mutated nucleic acid sequence of p57Kip2. Preferred mutant according to the present invention may be a dominant negative mutant.

Alternatively, in case decreased proliferation of beta-cells is desired, over-expression of p57Kip2 is advisable. Up-regulation of p57Kip2 may be achieved by transforming said cells with an expression vector comprising the sense nucleic acid sequence of the p57Kip2 and optionally an inducible promoter.

In yet another particularly preferred embodiment, the present invention relates to a method for the in vivo or ex vivo expansion of glucose regulated insulin-producing beta cells by down-regulation of p57Kip2 expression or activity. This method comprises the step of transforming said cells with an expression vector comprising an antisense nucleic acid sequence directed against the nucleic acid sequence encoding p57Kip2, or with expression vector comprising a mutated nucleic acid sequence of p57Kip2, and therefore down-regulation of the p57Kip2 and increased proliferation.

In yet another embodiment, the subject method can be applied to cell culture techniques, and in particular, may be employed to enhance the initial generation of prosthetic pancreatic tissue devices.

In an exemplary embodiment, the subject method can be used to augment production of prosthetic devices which require beta-islet cells, such as may be used in the encapsulation devices described in, for example, the Aebischer et al. U.S. Pat. No. 4,892,538, the Aebischer et al. U.S. Pat. No. 5,106,627, the Lim U.S. Pat. No. 4,391,909, and the Sefton U.S. Pat. No. 4,353,888. Early progenitor cells to the pancreatic islets are mutlipotential, and apparently coactive all the islet-specific genes from the time they first appear.

The phenotype of mature islet cells, however, is not stable in culture, as reappearance of embryonic traits in mature beta-cells can be observed. The expression vectors of the invention which modulate the expression or the activity of p57Kip2, can provide a means for more finely controlling the characteristics of a cultured tissue.

Furthermore, manipulation of the proliferative state of pancreatic tissue can be utilized in conjunction with transplantation of artificial pancreas so as to promote implantation, vascularization, and in vivo differentiation and maintenance of the engrafted tissue. For instance, manipulation of p57Kip2 function to affect cell proliferation can be utilized as a means of maintaining graft viability.

Therefore, a second aspect of the present invention relates to a recombinant glucose regulated insulin-producing beta cell or cell-line, transformed with an expression vector comprising any one of the sense, the antisense and mutated nucleic acid sequence of the p57Kip2. The recombinant beta-cell or cell-line according to the present invention is having controlled proliferation.

“Transformed cell” or “transfected cell” or cell line are terms used interchangeably herein. It is to be understood that such terms refer not only to the particular subject cells but to the progeny or potential progeny of such a cell. Because certain modifications may occur in a succeeding generation due to either mutation or environmental influences, such progeny may not, in fact, be identical to the parent cell, but are still included within the scope of the term as used herein. Thus, it will be appreciated that, as used herein, reference to “transfected cells” or “genetically modified cells” includes both the particular cell into which a vector or polynucleotide is introduced and progeny of that cell.

“Primary cells” are cells that have been harvested from the tissue of an organ.

The present invention further encompasses cell lines generated from the endocrine precursor cells of the human pancreas, and insulin-producing cell lines directly derived from human fetal pancreas, or fetal pancreas of any species. These cell lines, expanded by the methods of the invention, may be used for implantation. Thus, once the number of genetically modified cells has reached the desired amount for harvest, down-regulation of the p57Kip2 in the cells is removed, for example, due to the inducible promoter. These cells are then transplanted into the patient. Thus, regardless of the in vitro life span of the cell lines, the most preferred cell lines present non-dividing, preferably differentiated, human cell lines useful for transplantation.

It is to be appreciated that the beta-cell lines of the present invention, that were prepared by the method described herein and can be expanded as needed, may be used also as a powerful tool for basic research of different biological and physiological aspects on beta-cells.

This invention also pertains to a host cell transfected with the expression vector or DNA construct of the present invention. Ligating a polynucleotide sequence into a gene construct, such as an expression vector, and transforming or transfecting host cells with the vector are standard procedures used are well-known in the art.

Host cells suitable for modulating the expression or the activity of p57Kip2 can be selected, for example, from eukaryotic beta-cells. The transformed beta-cell or cell-line according to preferred embodiment of this aspect of the invention, may be a mammalian beta-cell.

Another possible type of target cell for transformation or transgene introduction according to the invention, is the embryonal stem cell (ES). ES cells are obtained from preimplantation embryos cultured in vitro and fused with embryos [Evans et al. Nature 292:154-156 (1981); Bradley et al. Nature 309:255-258 (1984); Gossler et al. PNAS 83:9065-9069 (1986); Robertson et al. Nature 322:445-448 (1986)]. Transgenes can be efficiently introduced into the ES cells by DNA transfection or by retrovirus-mediated transduction. Such transformed ES cells can thereafter be combined with blastocysts from a non-human animal. The ES cells thereafter colonize the embryo and contribute to the germ line of the resulting chimeric animal. For review see Jaenisch, R. (1988) Science 240:1468-1474.

In a specifically preferred embodiment, the beta-cell is transformed with an expression vector of the invention that further comprises an inducible promoter, a beta-cell specific transcriptional regulating sequence and optionally operably linked additional control, promoting and/or other regulatory elements. This expression vector may be a plasmid or a virus.

The beta-cell of the invention can express p57Kip2 in a modulated manner. This modulation, according to a preferred embodiment, may be down-regulation or up-regulation of the p57Kip2 expression or activity.

In a specifically preferred embodiment, the expression of p57Kip2 is down-regulated in the beta-cells of the invention. Accordingly, down-regulation of p57Kip2 expression may be achieved by transforming these cells with an expression vector comprising an anti-sense nucleic acid sequence directed against the nucleic acid sequence encoding p57Kip2. Down-regulation of p57Kip2 activity may be achieved by transforming these cells with an expression vector comprising a mutated nucleic acid sequence of p57Kip2. These expression vectors may optionally further include an inducible promoter. Thus, under suitable conditions, proliferation of such transformed cell would increase.

According to an alternative preferred embodiment, the expression of p57Kip2 is up-regulated in the beta-cells of the invention. Up-regulation of p57Kip2 expression may be achieved by transforming these cells with an expression vector comprising the sense nucleic acid sequence of the p57Kip2 and optionally an inducible promoter the beta-cell. Thus, such transformed cells would have decreased proliferation.

It is to be appreciated that creation of transgenic non-human animals carrying the expression vectors of the present invention is also contemplated within the scope of the present invention.

As used herein, a “transgenic animal” is any animal, preferably a non-human mammal, in which one or more of the cells of the animal contain a heterologous nucleic acid introduced by way of human intervention, such as by transgenic techniques well known in the art. The nucleic acid is introduced into the cell, directly or indirectly by introduction into a precursor of the cells, by way of deliberate genetic manipulation, such as by microinjection or by infection with a recombinant virus. The term genetic manipulation does not include classical cross-breeding, or in vitro fertilization, but rather is directed to the introduction of a recombinant DNA molecule. This molecule may be integrated within a chromosome, or it may be extra-chromosomally replicating DNA. In the typical transgenic animals which are within the scope of the present invention, the transgene causes cells to express a recombinant form of the sense or the anti-sense of the p57Kip2. Therefore, transgenic animals in which expression of the recombinant p57Kip2 gene is silent are particularly preferred. Transgenic animals also include both constitutive and conditional “knock out” animals. The “non-human animals” include vertebrates such as rodents, non-human primates, sheep, dog, pig, cow, chickens, amphibians, reptiles, etc. Preferred non-human animals are selected from the rodent family including rat and mouse, most preferably mouse. The term “chimeric animal” is used herein to refer to animals in which the recombinant gene is found, or in which the recombinant gene is expressed in some but not all cells of the animal. The term “tissue-specific chimeric animal” indicates that the p57Kip2 gene is over-expressed or silenced in some tissues but not others. This may be achieved by operably linking beta cell-specific sequences to the p57Kip sequences, as described hereinbefore.

As used herein, the term “transgene” means a nucleic acid sequence (the sense or the anti-sense of p57Kip2), which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cells into which it is inserted (e.g.) it is inserted at a location which differs from that of the natural gene or its insertion results in a knockout or other loss-of-function mutation. A transgene can include one or more transcriptional regulatory sequences, and preferably beta-cell specific elements and any other nucleic acid, that may be necessary for optimal expression of a selected nucleic acid.

In a further aspect, the present invention relates to a pharmaceutical composition for modulation of p57Kip2 expression. Such composition comprises as an active ingredient a therapeutically effective amount of the transformed beta-cells of the invention or of an expression vector comprising the sense or the antisense nucleic acid sequence of the p57Kip2. The composition of the invention further comprises pharmaceutically acceptable carriers.

In a preferred embodiment, the expression vector comprised in the composition of the invention further comprises an inducible promoter, a beta-cell specific transcriptional regulating sequence and optionally operably linked additional control, promoting and/or other regulatory elements. This expression vector may be a plasmid or virus.

Modulation of the expression of p57Kip2 by the pharmaceutical composition of the invention may be by down-regulation or up-regulation of the p57Kip2 expression.

In case down-regulation of p57Kip2 expression or activity is desired, the composition of the present invention may comprise as an effective ingredient an anti-sense nucleic acid sequence directed against the nucleic acid sequence encoding p57Kip2 and optionally an inducible promoter, or cells transformed with an expression vector comprising the same. For down-regulation of p57Kip2 activity the composition of the present invention may comprise as an effective ingredient a mutated nucleic acid sequence of p57Kip2 and optionally an inducible promoter, or cells transformed with an expression vector comprising the same.

Alternatively, when up-regulation of p57Kip2 expression is desired, the composition of the present invention may comprise as an effective ingredient a sense nucleic acid sequence of the nucleic acid sequence encoding p57Kip2 and optionally an inducible promoter, or cells transformed with an expression vector comprising the same.

The pharmaceutical compositions of the invention generally comprise a buffering agent, an agent which adjusts the osmolarity thereof, and optionally, one or more pharmaceutically acceptable carriers, excipients and/or additives as known in the art. Supplementary active ingredients can also be incorporated into the compositions. The carrier can be solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants.

As used herein “pharmaceutically acceptable carrier” includes any and all solvents, dispersion media, coatings, antibacterial and antifungal agents and the like. The use of such media and agents for pharmaceutical active substances is well known in the art. Except as any conventional media or agent is incompatible with the active ingredient, its use in the therapeutic composition is contemplated.

According to a preferred embodiment, the pharmaceutical composition of the invention is intended for the treatment of diabetes type I and/or diabetes type II and for disorders associated with increased beta-cell proliferation.

The inventor's finding of loss of p57KIP2 expression within the focal-HI lesion suggests that the gene is also imprinted in human beta-cells. This relatively simple immunohistologic stain can be used to confirm LOH of the maternal allele in these lesions and may be of use in differentiating focal-HI from other forms of hyperinsulinism. Similarly, the same stain may be useful in confirming LOH for this region in other tissues in diseases such as BWS and certain tumors such as Wilms' tumor [Hatada I, et al., Hum Mol Genet 5:783-8 (1996)], adrenocortical tumors [Bourcigaux N, et al., J Clin Endocrinol Metab 85:322-30 (2000)] and lung cancers [Kondo M, et al., Oncogene 12:1365-8 (1996)], as long as normal expression and imprinting is confirmed for each tissue type.

The finding of loss of p57^{KIP2} expression in focal-HI may explain the increased beta-cell proliferation in the adenomatous portion compared to unaffected pancreas and diffuse-HI [Kassem S A et al., Diabetes 49:1325-33 (2000); Sempoux C, Modern Pathology 11:444-9 (1998)]. However, the region of Ch11p lost in focal HI includes many other genes, some imprinted, that may play an additive or synergistic role in inducing beta-cell proliferation.

Therefore, it is to be appreciated that other possible, yet unidentified imprinted genes, located on the Ch11p which is lost in Focal Hi., are within the scope of the present invention. The use of such potential genes and genes such as the H19, in controlling cell proliferation of beta-cells, is also contemplated.

p57^{KIP2} positive cells tended to be more frequent outside the focal lesion compared to controls and diffuse-HI of the same age group, although this difference did not reach statistical significance. Beta-cells outside the lesion are exposed to hypoglycemia and high insulin concentration released from the lesion. This leads to suppressed metabolic activity and decreased cytoplasmic volume [Rahier J, et al., Histopathology 32:15-9 (1998)] and may also result in decreased proliferation mediated by high p57^{KIP2} expression.

It has been demonstrated by the present inventors that p57^{KIP2} is expressed and is paternally imprinted in human pancreatic beta-cells. Levels of expression do not appear to parallel changes in rates of beta-cell proliferation during development, whereas decreased expression in focal-HI is associated with increased rates of proliferation and increased IGF-II expression. Manipulation of p57KIP2 expression in beta-cells may provide a mechanism by which the rate of proliferation can be modulated, and thus, this gene may be a potential therapeutic target for reversing beta cell failure observed in diabetes.

Thus, the present invention further provides the use of a recombinant glucose regulated insulin-producing beta cell, transformed with an expression vector comprising an antisense nucleic acid sequence directed against the nucleic acid sequence encoding p57Kip2 according to the invention, in the preparation of pharmaceutical compositions for the treatment of diabetes type I and/or diabetes type II. Alternatively, the invention provides the use of a recombinant glucose regulated insulin-producing beta cell, transformed with an expression vector comprising a mutated nucleic acid sequence of p57Kip2 according to the invention, in the preparation of pharmaceutical compositions for the treatment of diabetes type I and/or diabetes type II.

In another specific embodiment, the invention relates to the use of an expression vector comprising the sense, the antisense or mutated nucleic acid sequence of the p57Kip2 for modulation of p57Kip2 expression or activity, in the preparation of pharmaceutical composition for the treatment of diabetes type I, diabetes type II or disorders associated with increased beta-cell proliferation.

Moreover, manipulation of the p57Kip2 expression may be useful for reshaping/repairing pancreatic tissue both in vivo and in vitro. In one embodiment, the present invention makes use of the apparent involvement of the p57Kip2 protein in controlling the proliferation of beta-cells for potential regulation of development of pancreatic tissue by the transformed beta-cells. For example, therapeutic compositions for modulating the expression of p57Kip2 can be utilized to preserve any beta-cells that have not been destroyed by diabetic or tumorogenic processes, as well as to induce regeneration of beta-cells so as to increase the islet mass. In general, the subject method can be employed therapeutically to regulate the pancreas after physical, chemical or pathological insult.

Where a nucleic acid sequence or the expression vector of the invention are used as the effective ingredient in the preparation of the composition of the invention, in vivo transformation should preferably be employed. In vivo transformation methods normally employ either a biological means of introducing the DNA into the target cells (e.g., a virus containing the nucleic acid sequence of interest) or mechanical means to introduce the DNA construct into the target cells (e.g., direct injection of DNA into the cells, liposome fusion, pneumatic injection using a “gene gun”). Generally the biological means used for in vivo transformation of target cells is a virus, particularly a virus which is capable of infecting the target cell, and integrating at least the DNA construct of interest into the target cell's genome, but is not capable of replicating. Such viruses are referred to as replication-deficient viruses or replication-deficient viral vectors. Alternatively, the virus containing the DNA construct of interest is attenuated, i.e. does not cause significant pathology or morbidity in the infected host (i.e., the virus is nonpathogenic or causes only minor disease symptoms).

Various mechanical means can be used to introduce the DNA construct of the invention directly into a pancreas of a mammalian subject. Direct administration of the DNA construct of the invention into the pancreas can be accomplished by cannulation of the pancreatic duct by, for example, duodenal intubation. Alternatively, administration of the virus containing the DNA construct of interest may be accomplished by intramuscular injection.

The nucleic acid sequence or expression vector of the invention may be naked (i.e., not encapsulated), provided as a formulation of DNA and cationic compounds (e.g., dextran sulfate), or may be contained within liposomes. Alternatively, the DNA construct of the invention can be pneumatically delivered using a “gene gun” and associated techniques which are well known in the art [Fynan et al. Proc Natl Acad Sci USA 90:11478-11482 (1993)].

A preferred approach for in vivo introduction of nucleic acid into a cell by use of a viral vector containing nucleic acid, e.g. the expression vector of the invention. Infection of cells with a viral vector has the advantage that a large proportion of the targeted cells can receive the nucleic acid. Additionally, molecules encoded within the viral vector, e.g., by a cDNA contained in the viral vector, are expressed efficiently in cells which has taken up the vector.

Expression vectors or constructs of the invention may be administered in any biologically effective carrier, e.g. any formulation or composition capable of effectively delivering to the cells in vivo. Approaches include insertion of the subject DNA constructs of the invention in different viral vectors or eukaryotic plasmids. Viral vectors transfect cells directly; plasmid DNA can be delivered with the help of, for example, cationic liposomes (lipofectin) or derivatized (e.g. antibody conjugated), polylysine conjugates, gramacidin S, artificial viral envelopes or other such intracellular carriers, as well as direct injection of the gene construct of CaPO₄ precipitation carried out in vivo. It will be appreciated that because transduction of appropriate target cells represents the critical first step in gene therapy, choice of the particular gene delivery system will depend on such factors as the phenotype of the intended target and the route of administration, e.g. locally or systemically. Furthermore, it will be recognized that the particular DNA construct provided for in vivo transduction of any homologous or heterologous coding sequence of interest, are also useful for in vitro transduction of cells.

Numerous viral vectors useful in in vivo transformation and gene therapy are known in the art, or can be readily constructed given the skill and knowledge in the art. Exemplary viruses include non-replicative mutants/variants of adenovirus, mumps virus, Lenti virus, retrovirus, adeno-associated virus, herpes simplex virus (HSV), cytomegalovirus (CMV) and vaccinia virus. Preferably, the replication-deficient virus is capable of infecting slowly replicating and/or terminally differentiated cells, since secratory glands (such as the pancreas) are primarily composed of these cell types. Thus, adenovirus is a preferred viral vector, since this virus efficiently infects slowly replicating and/or terminally differentiated cells. More preferably, the viral vector is specific or substantially specific for cells of the targeted pancreas gland.

In vivo gene transfer using biological means can be accomplished by administering the virus containing the desired nucleic acid sequence, to the mammalian subject either by an intraductal route, an oral route, or by injection. The amount of the DNA and/or the number of infectious viral particles effective to infect the target gland, transform a sufficient number of beta-cells and provide for transcription of therapeutic levels of the anti-sense sequence targeted against the p57Kip, or alternatively, expression of sufficient levels of the p57Kip, can be readily determined based upon such factors as the efficiency of the transformation in vitro, the levels of transcription achieved in vitro, and the susceptibility of the targeted gland cells to transformation.

As described above, the compositions of the invention can be administered in a variety of ways. By way of non-limiting example, in case nucleic acid sequence or expression vectors are used as the effective ingredient of the pharmaceutical composition of the invention, this composition may be delivered by injection.

The pharmaceutical forms suitable for injection use include sterile aqueous solutions or dispersions and sterile powders for the extemporaneous preparation of sterile injectable solutions or dispersions. In all cases the form must be sterile and must be fluid to the extent that easy syringability exists. It must be stable under the conditions of manufacture and storage and must be preserved against the contaminating action of microorganisms, such as bacteria and fungi.

The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above.

In the case of sterile powders for the preparation of the sterile injectable solutions, the preferred method of preparation are vacuum-drying and freeze drying techniques which yield a powder of the active ingredient plus any additional desired ingredient from a previously sterile-filtered solution thereof.

In yet a further aspect, the present invention relates to a method for treatment of diabetes type I and/or diabetes type II, in a subject having dysfunctional pancreatic beta-islet cells. According to this aspect, the method of the invention comprises administering to the subject in need a therapeutically effective amount of pharmaceutical composition comprising as an active ingredient a recombinant glucose regulated insulin-producing beta cell. This recombinant cell is transformed with an expression vector comprising an antisense nucleic acid sequence directed against the nucleic acid sequence encoding p57Kip2 according to the invention. Alternatively, the cell may be transformed with an expression vector comprising a mutated nucleic acid sequence of p57Kip2, for reducing the activity of p57Kip2.

As used herein, “effective amount” means an amount necessary to achieve a selected result. For example, an effective amount of the composition of the invention useful for controlling (reducing or increasing) the proliferation of said beta-cells. In case cells are administered, the effective amount is the amount usefull for producing sufficient amount of insulin and controlled glucose levels.

In a preferred embodiment, the method of the invention is intended for treating a mammalian subject, preferably, a human. And therefore, by “patient” or “subject in need” is meant any mammal for which gene therapy is desired, including human, bovine, equine, canine, and feline subjects, preferably, human patients.

The transformed cells of the invention may be administered directly to the animal to be treated, or it may be desirable to administer to the animal compositions comprising the transformed cells and it may be desirable to add acceptable carriers, adjuvants or diluents to the composition prior to its administration. Therapeutic formulations may be administered in any conventional dosage formulation. Formulations typically comprise at least one active ingredient, as defined above, together with one or more acceptable carriers thereof.

Each carrier should be pharmaceutically or veterinarily acceptable in the sense of being compatible with the other ingredients and not injurious to the treated animal. The formulations may conveniently be presented in unit dosage form and may be prepared by any methods well known in the art of pharmacy and veterinary.

Pharmaceutically acceptable carriers are well known to those skilled in the art and include, but are not limited to, 0.01-0.1M and preferably 0.05M phosphate buffer or 0.8% saline. Additionally, such pharmaceutically acceptable carriers may be aqueous or non-aqueous solutions, suspensions and emulsions. Examples for non-aqueous solvents are propylene glycol, vegetable oils such as olive oil, and injectable organic esters such as ethyl oleate. Aqueous carriers include water, alcoholic/aqueous solutions, emulsions or suspensions, including saline and buffer media.

Parenteral vehicles include fluid and nutrient replenishers, electrolyte replenishers such as those based on Ringers dextrose, and the like. Preservatives and other additives may also be present, such as, for example, antimicrobials, antioxidants, chelating agents, inert gases and the like.

In another specifically preferred embodiment, the method for treatment of diabetes type I and/or diabetes type II, in a subject having dysfunctional pancreatic beta-islets comprises administering to said subject in need a therapeutically effective amount of a pharmaceutical composition comprising as an active ingredient, an expression vector which comprises an antisense nucleic acid sequence directed against the nucleic acid sequence encoding p57Kip2 for down-regulation of p57Kip2 expression or an expression vector which comprises a mutated nucleic acid sequence of p57Kip2 for down-regulation of p57Kip2 activity and optionally an inducible promoter. Down-regulation of p57Kip2 expression or activity results in increased proliferation of the transformed beta-cells.

The subject method can be used as part of treatments for various forms of diabetes, as well as other pathologies resulting from direct physical/chemical damage to beta-cells which result in necrosis and loss of functional islet tissue. In diabetes mellitus, insulin secretion is either completely absent (IDDM) or inappropriately regulated (NIDDM). However, each is characterized by the presence of chronically elevated levels of blood glucose (hyperglycemia). The primary aim of treatment in both forms is the same, namely, the reduction of blood glucose levels to as near as normal as possible. For example, treatment of IDDM typically involves administration of replacement doses of insulin. In contrast, initial therapy for NIDDM may be based in part on therapies which include administration of hypoglycemic agents such as sulfonylurea, though insulin treatment in later stages of the disease may be required to effect normoglycemia. Accordingly, the present method can provide a means for controlling diabetogenous glycemic levels, by administration of the DNA construct of the invention, controlling the expression of p57Kip2 in beta-islet cells of the patient. For example, the anti-sense molecule which inhibits expression of p57Kip or the mutated p57Kip sequence which inhibits the activity of p57Kip, results in increasing beta-cell proliferation.

According to a further particular embodiment, the invention relates to a method for the treatment of a disorder of increased beta-cell proliferation in a subject. This method comprises administering to a subject in need a therapeutically effective amount of pharmaceutical composition comprising as an active ingredient an expression vector comprising the sense nucleic acid sequence encoding p57Kip2. This expression vector directs up-regulation of p57Kip2 expression and therefore, results in decreased proliferation of the target beta-cells.

An increased proliferation disorder or a hyperproliferative disorder is a disorder wherein cells present in a subject suffering from the disorder proliferate at an abnormally high rate, which is a cause for the disorder. In one non-limiting embodiment, the increased beta-cell proliferation disorder is hyperinsulinism, which is a non-neoplastic defect in beta-cell function.

In another non-limiting exemplary embodiment, the present method can be used in the treatment of hyperplastic and neoplastic disorders effecting pancreatic tissues, particularly those characterized by aberrant proliferation of beta-cells. For instance, pancreatic tumors, such as islet tumors (e.g., insuliomas), are marked by overproduction of insulin (i.e., hyperinsulinemia) which can cause hypoglycemic conditions in a patient. Hypoglycemia can result from any one of a number of different disorders which result in raised plasma insulin levels, including other beta-cell abnormalities, as well as endocrinopathies, sepsis (including malaria), congestive cardiac failure, hepatic and renal insufficiencies, various genetic abnormalities of metabolism, and exogenous toxins (such as alcohol).

According to the present invention, these conditions can be treated by administering therapeutic amounts of expression vector comprising the sense nucleic acid of the p57Kip2. Such treatment up-regulates the transcription of p57Kip and leads to decreased proliferation of the cells.

Introduction of the DNA construct or the expression vector of the present invention into the pancreatic cell can be accomplished by various methods well known in the art. For example, transformation of pancreatic cells can be accomplished by administering the DNA of interest directly to the mammalian subject (in vivo gene therapy, as detailed herein before), or to an in vitro culture of a biopsy of pancreatic cells which are subsequently transplanted into the mammalian subject after transformation (ex vivo gene therapy).

Therefore, the present invention further provides a method for ex vivo treating an individual suffering from diabetes type I or diabetes type II. Such method comprises the steps of: (a) providing an expression vector comprising an antisense nucleic acid sequence directed against the nucleic acid sequence encoding p57Kip2 or a mutated nucleic acid sequence of p57Kip2 for modulation of p57Kip2 expression or activity; (b) obtaining cells from an in individual suffering from diabetes type I or diabetes type II, and optionally culturing said cells under suitable conditions; (c) transforming the cells obtained in step (b) with the expression vector provided in (a); (d) in vitro expanding said transformed cells under suitable conditions; and (e). re-introducing said cells obtained in (d) into said individual.

Alternatively, expansion of the transformed beta-cells may be performed in vivo, under certain conditions suitable for inducing the expression of the antisense nucleic acid sequence directed against the nucleic acid sequence encoding p57Kip2 or the mutated nucleic acid sequence of p57Kip2.

As indicated above, the pancreatic cells of a patient may be transformed ex vivo by collecting a biopsy of the pancreas tissue, and establishing a primary culture of these pancreatic cells. Methods of growing cells from this tissue in vitro are well known in the art. Methods for separation of cells from tissue (see, for example, Amsterdam et al., J Cell Biol 63:1057-1073, (1974), and methods for culturing cells in vitro are well known in the art.

The pancreatic cells in the in vitro culture are transformed using various methods known in the art. For example, transformation can be performed by calcium or strontium phosphate treatment, microinjection, electroporation, lipofection, or viral infection.

After expansion of the transformed pancreatic cells in vitro, the cells are implanted into the mammalian subject, preferably into the pancreas from which the cells were originally derived, by methods well known in the art. Preferably the cells are implanted in an area of dense vascularization, and in a manner that minimizes evidence of surgery in the subject. The engraftment of the implant of transformed pancreatic cells is monitored by examining the mammalian for classic signs of graft rejection, i.e., inflammation and/or exfoliation at the site of the implantation, and fever.

Cells genetically modified by the nucleic acid sequences or expression vectors of the invention, will be used for cell transplantation therapies. These cells are expanded by the method of the invention and when enough cells are available, their growth is stopped and these cells are transplanted into a patient, e.g., to replace the destroyed or malfunctioning cells in the patient or to produce the desirable gene products. The genetically modified cells are preferably of the same species as the host into which they will be transplanted. Generally, mammalian target cells are used for treating mammalian subjects. Thus, in the case of a human patient, the cells are preferably human.

The target cells can be adult (e.g. cadavar donor beta-cells) or precursor cells. Precursor cells are cells which are capable of differentiating, e.g., into an entire organ or into a part of an organ. Such cells are capable of generating or differentiating to form a particular tissue (e.g., muscle, skin, heart, brain, uterus, and blood). Examples of precursor cells are endocrine precursor cells and fetal cells. Fetal cells are readily obtained and capable of further growth. In the case of recombinant retroviruses, fetal cells are still capable of division and can therefore serve as targets for these viruses.

In a preferred embodiment, the donor target cells are from human pancreas, which may be either fetal or adult. In one embodiment adult human islets may be used [Wang et al., Diabetes 45 supplement 2:285A5 (1996)]. Preferably, the cells are purified beta-cells that may be separated from non beta-cells (such as delta and PP) found in human pancreas on the basis of cell properties, such as the ability of beta-cells to accumulate flavin adenine dinucleotide (FAD) when incubated in a medium containing a low concentration of glucose.

Alternatively, the pancreatic cells are transformed in vivo by either mechanical means (e.g., direct injection of the DNA of interest into or in the region of the pancreas or lipofection) or by biological means (e.g., infection of a pancreas with a non-pathogenic virus, preferably a non-replicative virus, containing the DNA construct of interest).

Thus, as described above, the expression vector of interest can be delivered to the subject or the in vitro cell culture as, for example, purified DNA, in a viral vector (e.g., adenovirus, mumps virus, retrovirus), a DNA- or RNA-liposome complex, or by utilizing cell-mediated gene transfer. Further, the vector, when present in non-viral form, may be administered as a DNA or RNA sequence-containing chemical formulation coupled to a carrier molecule which facilitates delivery to the host cell. Such carrier molecules can, for example, include an antibody specific to an antigen expressed on the surfaces of the targeted pancreatic cells, or some other molecule capable of interaction with a receptor associated with pancreatic cells. Generally, transformation is accomplished by either infection of the pancreatic cells with a virus, preferably a replication-deficient virus, containing the DNA construct of interest, or by a non-viral transformation method, such a direct injection of the DNA into or near the target salivary gland cell, lipofection, “gene gun”, or other methods well known in the art. The preferred methodology is dependent upon whether the gene transfer is performed ex vivo or in vivo.

By “pancreatic” is meant of pancreas, by “pancreas” is meant a large, elongated, racemose gland situated transversely behind the stomach, between the spleen and duodenum. The pancreas is composed of an endocrine portion (the pars endocrina) and an exocrine portion (the pars exocrina). The pars endocrina, which contains the islets of Langerhans, produces and secretes proteins, including insulin, directly into the blood stream. The pars exocrina contains secretory units and produces and secretes a pancreatic juice, which contains enzymes essential to protein digestion, into the duodenum.

Disclosed and described, it is to be understood that this invention is not limited to the particular examples, process steps, and materials disclosed herein as such process steps and materials may vary somewhat. It is also to be understood that the terminology used herein is used for the purpose of describing particular embodiments only and not intended to be limiting since the scope of the present invention will be limited only by the appended claims and equivalents thereof.

It must be noted that, as used in this specification and the appended claims, the singular forms “a”, “an” and “the” include plural referents unless the content clearly dictates otherwise.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The following examples are representative of techniques employed by the inventors in carrying out aspects of the present invention. It should be appreciated that while these techniques are exemplary of preferred embodiments for the practice of the invention, those of skill in the art, in light of the present disclosure, will recognize that numerous modifications can be made without departing from the spirit and intended scope of the invention.

### EXAMPLES

Experimental Procedures

Antibodies

Rabbit anti p57^{KIP2}—was purchased from Santa Cruz.

Guinea Pig anti-insulin was purchased from Dako.

Rabbit anti-glucagon, Rabbit anti-SMS and Rabbit anti-PP where purchased from DPC (Diagnostic Products corporation).

Mouse anti-IGF-II was purchased from Upstate.

Patients: Archival tissues from 15 pancreatectomized HI patients were obtained from 5 clinical centers (Table 1). In all, the diagnosis of HI was made according to accepted criteria (Aynsley-Green A, Dev Med Child Neurol 23:372-9 (1981); Landau H, et al., Pediatrics 70:440-6 (1982)]. Eleven males and 4 females, age range 2 weeks to 13 months, were included in the study. Eleven patients had focal disease, and 4 had a diffuse form of HI. Twelve of these subjects were previously reported (Table 1).

Controls: Fifteen control pancreatic samples were included in the study. Twelve were obtained from autopsies carried out between 1988-1998 consisting of 7 males and 5 females aged 17 weeks gestation to 3 years old. These samples consist of a random subgroup of the previously published control population [Kassem S A, et al., Diabetes 49:1325-33 (2000)]. All fetuses and infants died as a result of diseases not related to the pancreas, and in all, autopsies were done for medical reasons according to accepted procedures at each individual institution. All dysmorphic subjects were excluded, as were subjects with known chromosomal abnormalities or genetic syndromes. Only subjects in whom the autopsy was performed within 24 h of death were included. Adult control pancreas samples were obtained from two pancreas donors and from a patient who underwent partial pancreatectomy for insulinoma. All samples were stained with H&E and screened for adequate quantity of tissue, normal morphology and good tissue preservation.

TABLE 1
Clinical characteristics of HI patients

| | | | | | | | |
|---|---|---|---|---|---|---|---|
| | | Birth | | Age | | | |
| Pt. | | Weight | Age of onset | surgery | at Postoperative | Paternal | Maternal |
| # | Sex | (kg) | (months) | (months) | status | Mutation | mutation |

Diffuse-HI

| | | | | | | | |
|---|---|---|---|---|---|---|---|
| 1 | F | 4.1 | 1.25 | 1.5 | Hypoglycemic | Int 32-3c | N188S |
| | | | | | | to g | |
| 2 | M | 3.6 | Birth | 1.6 | Diabetes | delcP317 | delcP317 |
| 3 | M | 5.04 | Birth | 3.25 | Hypoglycemic | Kir Y12X | Kir Y12X |
| 4 | M | 4.4 | Birth | 13 | Hypoglycemic | Int 32-9 | delF1388 |
| | | | | | | g to a | |

Focal-HI

| | | | | | | | |
|---|---|---|---|---|---|---|---|
| 5 | M | 5.36 | Birth | 0.5 | Euglycemic | Int 32-9 | None found |
| | | | | | | g to a | |
| 6 | M | 3.19 | Birth | 2 | Euglycemic | R1494Q | None found |
| 7 | F | 3.3 | 5 | 6 | Euglycemic | No DNA | |
| 8 | F | 3.25 | Birth | 0.833 | Euglycemic | None found* | None found |
| 9 | M | 4.18 | Birth | 1.25 | Diabetes | None found | None found |
| 10 | M | 3.61 | Birth | 5.5 | Euglycemic | None found | None found |
| 11 | F | 3 | Birth | 12 | No Data | No DNA | |
| 12 | M | 4 | Birth | 1.5 | Diabetes | None found | None found |
| 13 | M | 3.9 | Birth | 2 | Euglycemic | No DNA | |
| 14 | M | 3.8 | Birth | 3 | Diabetes | No DNA | |
| 15 | M | 3.63 | 10 | 11 | Euglycemic | A1493T | None found |

Subjects # 1-13 were evaluated for p57^{KIP2} expression whereas subjects # 8-15 were evaluated for IGF-II expression. All patients except for #s 12-14 were previously reported [Kassem SA, ibid. (2000)].
*For samples in which no mutation was found, only part of the coding sequence of SUR1 and Kir6.2 were thus far sequenced. It is likely, therefore, that mutations will be identified in the future.

Immunohistochemistry: Five micron sections were prepared from archival paraffin-embedded tissue, placed on SuperFrost Plus glass slides (Menzel-Glaser, Germany), and left to dry at 37° C. overnight. Slides were deparaffinized in xylene, rehydrated in serial concentrations of alcohol (100, 90, and 80%) and double distilled water. Antigen retrieval was carried out as described by Cattoretti [Cattoretti G, et al., Journal of Pathology 168:357-63 (1992)]. Briefly, slides were microwaved in 0.01M citrate buffer (pH 6) for 3 min. at full power until boiling, and for 15 min. at 20% power. Slides were left to cool at room temperature (RT) for 30 min. Slides were blocked by non-immune serum for 10 min. at RT prior to application of each primary antibody.

p57^{KIP}2—hormone double staining: Slides were double stained for p57^{KIP2} and each of the 4 major pancreatic hormones (insulin, glucagon, somatostatin, and pancreatic polypeptide). Antibodies, incubation times, detection systems and substrates are listed in Table 2. To prevent cross reactivity of the 2 detection systems, avidin-biotin blocking kit (Zymed cat#00-4303) was used prior to incubation with anti-hormone antibody. As negative control, slides underwent the same procedure but were incubated with PBS without anti-p57^{KIP2} antibody. Each batch included a negative control.

IGF-II/insulin: Sections were double-stained for IGF-II and insulin. Antibodies, incubation times, detection systems and substrates are listed in Table 2. Cross reactivity of the anti-IGF-II antibody with proinsulin or insulin was excluded by pre-absorbing the antibody with the 2 peptides overnight, a procedure that did not affect the intensity of the stain. Pretreatment, incubation times and conditions were similar for all slides in each step described.

TABLE 2
Materials and incubation details for immunohistology

| | | | | | |
|---|---|---|---|---|---|
| | | | Incubation | | |
| Primary | | Concen- | Time & | | |
| Antibody | Supplier | tration | Temp. | Detection System | Substrate |

p57^{KIP2} Expression Study

| | | | | | |
|---|---|---|---|---|---|
| Rb anti | Santa | 1\500 | 1 h 37° C. | Strptavidin Biotin | DAB- |
| p57^{KIP2} | Cruz | | | Peroxidase | black |
| GP anti- | Dako | 1\100 | 1 h 37° C. | Strptavidin Biotin | FR |
| insulin | | | | Alk. Phos. | |
| Rb anti- | DPC | As | 1 h 37° C. | Strptavidin Biotin | FR |
| glucagon | | supplied | | Alk. Phos. | |
| Rb anti- | DPC | As | 1 h 37° C. | Strptavidin Biotin | FR |
| SMS | | supplied | | Alk. Phos. | |
| Rb anti-PP | DPC | As | 1 h 37° C. | Strptavidin Biotin | FR |
| | | supplied | | Alk. Phos. | |

IGF-II Quantification Study

| | | | | |
|---|---|---|---|---|
| Ms anti- | Upstate | 1\100 | 1 h 37° C. | Gt anti Ms CY5 conjugate |
| IGF-II | | | | |
| GP anti- | Dako | 1\100 | 1 h 37° C. | Rb anti GP FITC conjugate |
| insulin | | | | |

Abbreviations: Rb = rabbit; GP = guinea pig; Ms = mouse; Gt = goat; DPC = Diagnostic Products Corporation; DAB = diaminobenzidine tetrachloride; FR = fast red.

Quantification:

p57^{KIP2}/hormone: All slides were coded, and at least 1000 hormone-positive cells were counted under high magnification (×400). The frequency of p57^{KIP2}/hormone-positive cells was determined and expressed as percent of hormone positive cells (mean±standard error).

IGF-II/Insulin: Eleven different fields were assessed under high magnification (×400). Two images were produced from each field, using different filters in the same settings of microscope and camera (L-600, Coolpix 950, respectively, Nikon)

Images were analyzed using Image-Pro Plus software (Media Cybernetics). Total stained area was expressed in pixels and total integrated optic density was expressed in arbitrary optic density units. Beta-cell IGF-II protein content was expressed as a ratio of IGF-II IOD/Insulin-stained area. Counting criteria and software settings were identical for all slides.

Statistical analysis: Results for p57^{KIP2} expression in different age-groups were analyzed using the Kruskal-Wallis Nonparametric ANOVA test whereas the HI groups were compared to controls using the Mann-Whitney test. The Wilcoxon paired non-parametric test was used to compare IGF-II expression inside and outside the lesion in focal-HI.

### Example 1

p57^{KIP2} Expression

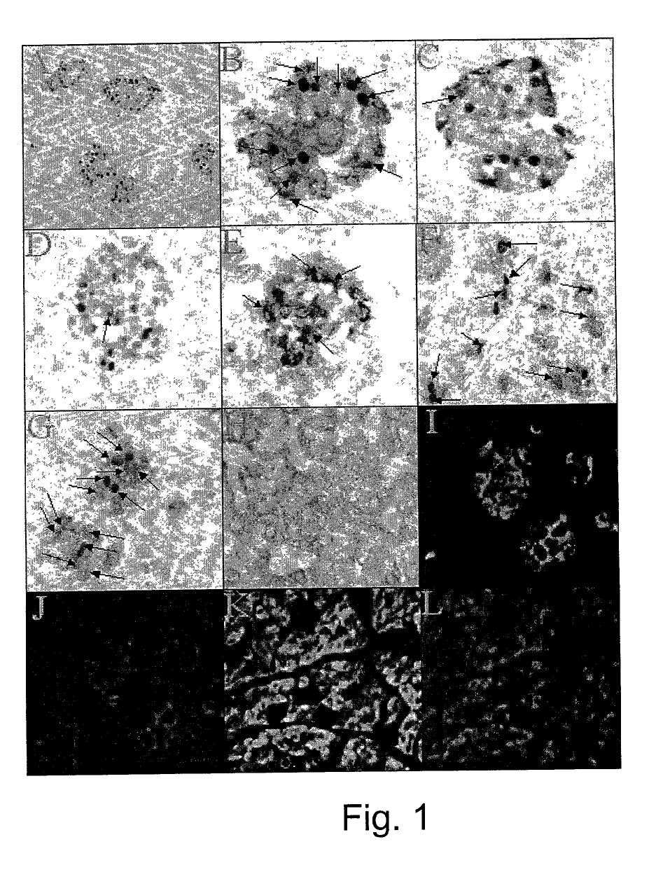

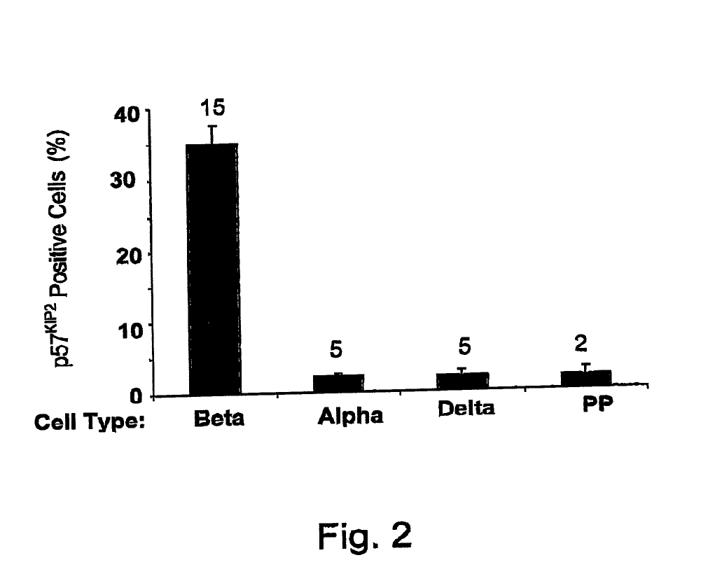

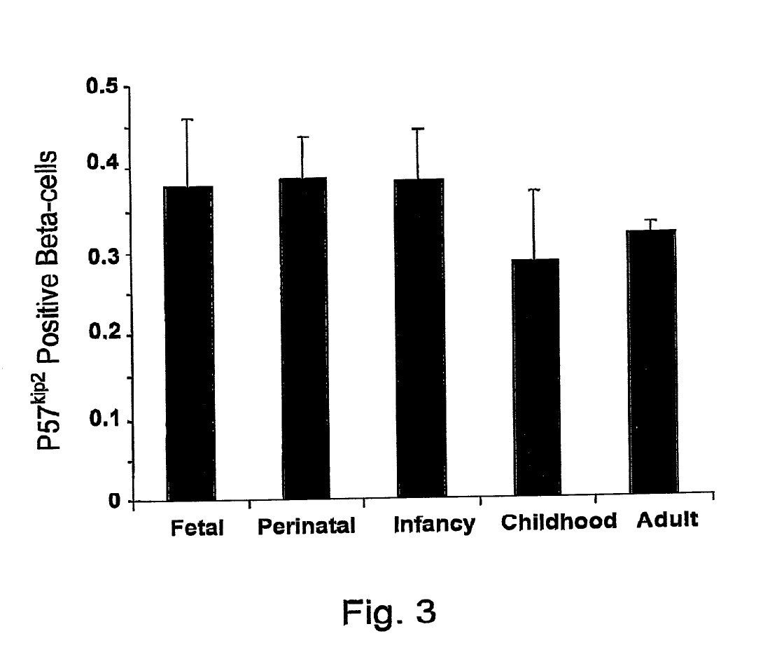

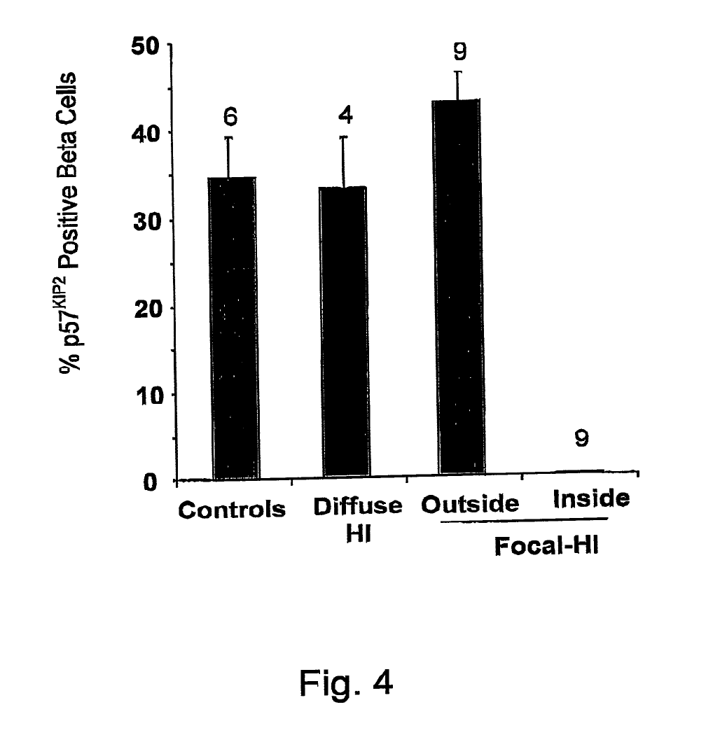

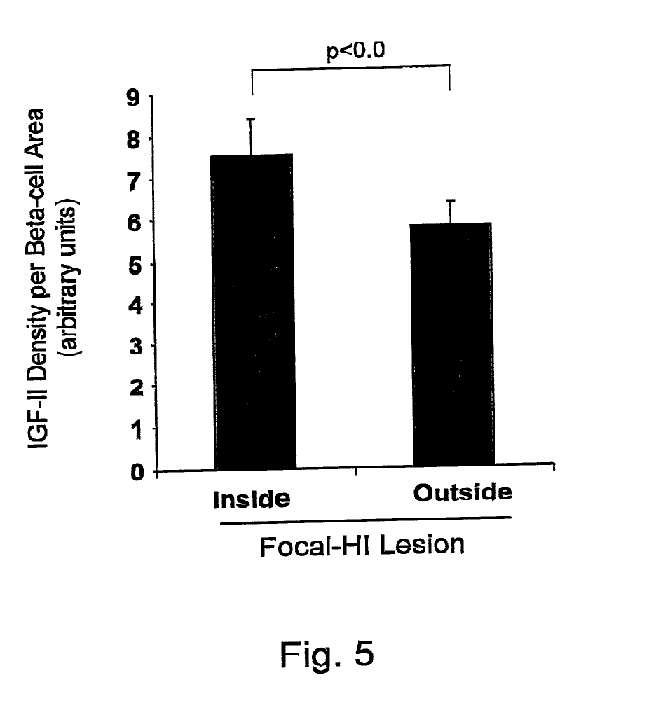

In order to analyze the expression pattern of p57^{KIP2} in the endocrine portion of the pancreas, immunohystochemical staining for p57^{KIP2}/hormones was performed as described herein above. p57^{KIP2} expression was demonstrated as dark brown nuclear staining while pancreatic hormones were stained red in cell cytoplasm (FIGS. 1A-H). p57^{KIP2} was specifically localized to the endocrine portion of pancreas with a clear islet-specific distribution. Very few p57^{KIP2} positive cells were seen in the acinar tissue (FIG. 1A). In the normal pancreas, beta-cells demonstrated the highest frequency of p57^{KIP2} expression (34.9±2.7%,), whereas other islet cell types stained for p57^{KIP2} with much lower frequency (˜1-3%) (FIGS. 1B-D and FIG. 2). No significant change in p57^{KIP2} positive beta cell proportion was observed during the different developmental stages of the human pancreas (FIG. 1E and FIG. 3).

The finding that p57^{KIP2} expression does not change during different stages of development is unexpected, since the proportion of beta-cells undergoing proliferation does change during fetal development as previously reported [Kassem S A, ibid. (2000)]. However, the highest proliferation frequency was reported to be about 5% at the gestational week 17. Since only 30-40% of beta-cells are p57^{KIP2} positive, it is likely that the methods used are not sufficiently sensitive to detect small absolute differences in the low proportion of cells undergoing proliferation at the different developmental stages.

Focal-HI is caused by specific loss within affected beta-cells of a portion of the maternal allele of Ch11p which contains the p57^{KIP2} gene [Fournet J C, et al., Endocrinologie 59:485-91 (1998); Fournet J C, et al., Horm Res 53:2-6 (2000)]. p57KIP2 has been shown to be paternally imprinted in several tissues [Matsuoka S, et al. Proc. Natl. Acad. Sci. U.S.A. 93:3026-30, (1996)].

The percentage of p57^{KIP2} positive beta-cells in diffuse-HI was similar to that in the controls (FIG. 1F and FIG. 4). Complete loss of p57KIP2 staining was clearly demonstrated inside affected area of focal-HI (FIGS. 1G-H). Interestingly, a tendency toward increased p57^{KIP2} expression was observed in beta-cells outside the affected area of focal HI compared to diffuse-HI and controls, although this did not reach statistical significance (FIG. 4).

### Example 2

IGF-II Expression

IGF-II is located in the same region on chromosome 11 but is maternally imprinted and it has been associated with increased beta cell proliferation [Petrik J, Endocrinology 140:2353-63 (1999)]. Since the increased proliferation previously documented in focal-HI could be due to increased expression of the maternally imprinted IGF-II gene, the IGF-II protein content of beta-cells inside and outside of the lesion, was next quantitated. The inventors developed a method using quantitative image analysis of immunofluorescence to estimate the IGF-II content of affected and unaffected beta-cells in focal-HI. By comparing IGF-II optical density (IOD) to insulin area, the inventors obtained an estimate of the quantity of IGF-II protein as a function of beta-cell area. Since it previously show that IGF-II is expressed exclusively in beta-cells, this calculation defines the amount of protein within the beta-cells. Insulin content (as defined by insulin IOD) was not used since this reflects the secretory state of the beta-cells which clearly differs inside and outside the lesion. Beta-cells that underwent complete degranulation were not included in this calculation, however examination of the sections indicated that very few if any of the cells within the lesion are completely degranulated and those outside the lesion are uniformly heavily granulated, reflecting the suppressed secretion in these functionally normal cells.

As shown in FIG. 1, IGF-II staining was identified exclusively in beta-cell cytoplasm both inside and outside the focal lesion (FIGS. 1I-L). Outside the lesion, the area stained with IGF-II was a subset of the insulin-stained area consisting of approximately 27% of the insulin area. In normal beta-cells from the same age group, a similar IGF-II distribution was seen (data not shown). In order to quantify the amount of IGF-II within the beta-cell mass, the intensity of IGF-II staining was expressed as a ratio between IGF-II integrated optical density and insulin stained area. In focal-HI, IGF-II staining within the focal lesion was slightly increased when compared to that outside of the lesion in the same patient (7.5±0.9 vs 5.7±0.6 arbitrary units, p<0.04; FIG. 5).

This finding of increased IGF-II within the focal lesion, relative to outside the lesion, supports the hypothesis that IGF-II may be involved in regulation of focal proliferation. The mechanism causing this increased IGF-II expression is unknown. Paternal disomy has been documented in focal-HI [Fournet J-C, et al., Am J Pathol 158:(in press) (2001)], and this increase in gene dosage may be responsible for increased expression. Alternatively, H19, a paternally imprinted gene thought to regulate IGF-II expression [Li M, et al., Clinical Genetics 53:165-70 (1998)] may play a critical role, however H19 expression and imprinting in beta-cells has not yet been proven. It is also possible that p57^{KIP2} may have a direct effect on IGF-II expression, although such a connection has not yet been established. Furthermore, the relatively small absolute difference raises the possibility that this may be secondary phenomenon.

1. A method for controlling proliferation of glucose regulated insulin-producing beta cells by modulating any one of the expression and the activity of the cyclin-dependent kinase inhibitor p57Kip2, which method comprises the step of transforming said cells with an expression vector comprising any one of the sense, the antisense and mutated nucleic acid sequence of the p57Kip2.

2. The method of claim 1, wherein said expression vector further comprises an inducible promoter, a beta-cell specific transcriptional regulating sequence and optionally operably linked additional control, promoting and/or other regulatory elements.

3. The method according to claim 2, wherein said expression vector is any one of plasmid and virus.

4. The method according to claim 1, wherein said modulation of the expression of p57Kip2 is any one of down-regulation and up-regulation of the p57Kip2 expression and/or activity.

5. The method according to claim 4, wherein said down-regulation of p57Kip2 expression is achieved by transforming said cells with an expression vector comprising an anti-sense nucleic acid sequence directed against the nucleic acid sequence encoding p57Kip2 and optionally an inducible promoter, which down-regulation of p57Kip2 expression results in increased proliferation of said transformed cell.

6. The method according to claim 4, wherein said down-regulation of p57Kip2 activity is achieved by transforming said cells with an expression vector comprising a mutated nucleic acid sequence encoding p57Kip2 and optionally an inducible promoter, which down-regulation of p57Kip2 activity results in increased proliferation of said transformed cell.

7. The method according to any one of claims 5 and 6, for the in vivo or ex vivo expansion of glucose regulated insulin-producing beta-cells by down-regulation of any one of p57Kip2 expression and activity, which method comprises the step of transforming said cells with an expression vector comprising any one of mutated and antisense nucleic acid sequence directed against the nucleic acid sequence encoding p57Kip2.

8. The method according to claim 4, wherein said up-regulation of p57Kip2 expression is achieved by transforming said cells with an expression vector comprising the sense nucleic acid sequence of the p57Kip2 and optionally an inducible promoter, which up-regulation of p57Kip2 expression results in decreased proliferation of said transformed cell.

9. A recombinant glucose regulated insulin-producing beta cell or cell-line, transformed with an expression vector comprising any one of the sense, the antisense and mutated nucleic acid sequence of the p57Kip2.

10. The beta-cell according to claim 9, wherein said cell is a mammalian beta-cell.

11. The beta-cell according to claim 10, wherein said expression vector further comprises an inducible promoter, a beta-cell specific transcriptional regulating sequence and optionally operably linked additional control, promoting and/or other regulatory elements.

12. The beta-cell according to claim 11, wherein said expression vector is any one of plasmid and virus.

13. The beta-cell according to claim 9, having modulated expression and/or activity of p57Kip2, wherein said modulation is any one of down-regulation and up-regulation of the p57Kip2 expression and/or activity.

14. The beta-cell according to claim 13, wherein said down-regulation of p57Kip2 expression achieved by transforming said cells with an expression vector comprising an anti-sense nucleic acid sequence directed against the nucleic acid sequence encoding p57Kip2 and optionally an inducible promoter.

15. The beta-cell according to claim 13, wherein said down-regulation of p57Kip2 activity is achieved by transforming said cells with an expression vector comprising a mutated nucleic acid sequence encoding p57Kip2 and optionally an inducible promoter.

16. The beta-cell according to claim 13, wherein said up-regulation of p57Kip2 expression is achieved by transforming said cells with an expression vector comprising the sense nucleic acid sequence of the p57Kip2 and optionally an inducible promoter.

17. A pharmaceutical composition for modulation of any one of p57Kip2 expression and activity comprising as an active ingredient a therapeutically effective amount of any one of the transformed beta-cells according to any one of claims 9 to 16, and expression vector comprising any one of the sense, the antisense and mutated nucleic acid sequence of the p57Kip2, and a pharmaceutically acceptable carrier.

18. The pharmaceutical composition according to claim 17, wherein said expression vector further comprises an inducible promoter, a beta-cell specific transcriptional regulating sequence and optionally operably linked additional control, promoting and/or other regulatory elements.

19. The pharmaceutical composition according to claim 18, wherein said expression vector is any one of plasmid and virus.

20. The pharmaceutical composition according to claim 17, wherein said modulation of the expression of p57Kip2 is any one of down-regulation and up-regulation of the p57Kip2 expression and/or activity.

21. The pharmaceutical composition according to claim 20, wherein said down-regulation of p57Kip2 expression is achieved by transforming said cells with an expression vector comprising an anti-sense nucleic acid sequence directed against the nucleic acid sequence encoding p57Kip2 and optionally an inducible promoter.

22. The pharmaceutical composition according to claim 20, wherein said down-regulation of p57Kip2 activity is achieved by transforming said cells with an expression vector comprising a mutated nucleic acid sequence of the p57Kip2 and optionally an inducible promoter.

23. The pharmaceutical composition according to claim 20, wherein said up-regulation of p57Kip2 expression is achieved by transforming said cells with an expression vector comprising the sense nucleic acid sequence of the p57Kip2 and optionally an inducible promoter.

24. The pharmaceutical composition according to any one of claims 17 and 21, for the treatment of any one of diabetes type I and diabetes type II.

25. A method for treatment of any one of diabetes type I and diabetes type II, in a subject having dysfunctional pancreatic beta-islet cells comprising administering to said subject in need a therapeutically effective amount of a pharmaceutical composition comprising as an active ingredient a recombinant glucose regulated insulin-producing beta cell, transformed with an expression vector comprising an antisense nucleic acid sequence directed against the nucleic acid sequence encoding p57Kip2 according to claim 14.

26. A method for treatment of any one of diabetes type I and diabetes type II, in a subject having dysfunctional pancreatic beta-islet cells comprising administering to said subject in need a therapeutically effective amount of a pharmaceutical composition comprising as an active ingredient a recombinant glucose regulated insulin-producing beta cell, transformed with an expression vector comprising a mutated nucleic acid sequence the p57Kip2 according to claim 15.

27. A method for treatment of any one of diabetes type I and diabetes type II, in a subject having dysfunctional pancreatic beta-islet cells, comprising administering to said subject in need a therapeutically effective amount of a pharmaceutical composition comprising as an active ingredient an expression vector comprising an antisense nucleic acid sequence directed against the nucleic acid sequence encoding p57Kip2 for down-regulation of p57Kip2 expression and optionally an inducible promoter.

28. A method for treatment of any one of diabetes type I and diabetes type II, in a subject having dysfunctional pancreatic beta-islet cells, comprising administering to said subject in need a therapeutically effective amount of a pharmaceutical composition comprising as an active ingredient an expression vector comprising a mutated nucleic acid sequence of p57Kip2 for down-regulation of p57Kip2 activity and optionally an inducible promoter.

29. A method for treatment of a disorder of increased beta-cell proliferation in a subject, comprising administering to said subject in need a therapeutically effective amount of pharmaceutical composition comprising as an active ingredient, an expression vector comprising the sense nucleic acid sequence encoding p57Kip2 for up-regulation of p57Kip2 expression.

30. The method according to any one of claims 25 to 29, wherein said subject is a mammalian subject.

31. The method according to claim 30, wherein said mammal is human.

32. A method for ex-vivo treating an individual suffering from any one of diabetes type I and diabetes type II, comprising:
(a) providing an expression vector comprising any one of mutated and antisense nucleic acid sequence of the p57Kip2 for modulation of any one of p57Kip2 expression and activity;
(b) obtaining cells from an in individual suffering from said of diabetes type I or diabetes type II, and optionally culturing said cells under suitable conditions;
(c) transforming the cells obtained in step (b) with the expression vector provided in (a);
(d) in vitro expanding said transformed cells under suitable conditions; and
(e) re-introducing said cells obtained in (d) into said individual.

33. A method for ex-vivo treating an individual suffering from any one of diabetes type I and diabetes type II, comprising:
(a) providing an expression vector comprising any one of antisense and mutated nucleic acid sequence of the p57Kip2 for modulation of any one of p57Kip2 expression and activity;
(b) obtaining cells from an in individual suffering from said of diabetes type I or diabetes type II, and optionally culturing said cells under suitable conditions;
(c) transforming the cells obtained in step (b) with the expression vector provided in (a);
(d) re-introducing said cells obtained in (c) into said individual; and
(e) in vivo expanding said transformed cells under suitable conditions.
